(19) United States
(12) Patent Application Publication
(10) Pub. No.: US 2008/0091015 A1
(43) Pub. Date: Apr. 17, 2008

(54) USE OF SQUARAINE DYES TO VISUALIZE PROTEIN DURING SEPARATIONS

(75) Inventors: Thomas R. Berkelman, Oakland, CA (US); Sergiy M. Yarmoluk, Kyiv (UA); Vladyslava B. Kovalska, Kyiv (UA); Mykhaylo Yu Losytskyy, Kyiv (UA); Kateryna D. Volkova, Kyiv (UA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 11/842,779

(22) Filed: Aug. 21, 2007

Related U.S. Application Data
(60) Provisional application No. 60/823,886, filed on Aug. 29, 2006.

(51) Int. Cl. C07D 239/54; C07D 209/12

(52) U.S. Cl. 544/301; 548/455

(57) ABSTRACT

Squaraine dyes are incorporated into a separation medium in which protein or polypeptide mixtures are separated, which medium also contains a detergent that forms a complex with the polypeptides or proteins. The dyes, upon excitation in the separation medium, exhibit a heretofore unrecognized selectivity in their fluorescent emissions by emitting a signal only when the dye molecules are associated with complexes of protein (or polypeptide) and detergent molecules, despite the additional presence of dyes in the bulk of the separation medium. The dyes are thus able to indicate the presence and locations of proteins or polypeptides in the separation medium without the need for removing unassociated dyes from the medium.

### CROSS-REFERENCE TO RELATED APPLICATION

This application claims the benefit of U.S. Provisional Patent Application No. 60/823,886, filed Aug. 29, 2006, the contents of which are incorporated herein by reference in their entirety.

### BACKGROUND OF THE INVENTION

1. Field of the Invention

This invention resides in the field of protein labeling and detection.

2. Description of the Prior Art

The use of lipophilic dyes for labeling proteins by associative interactions that do not involve covalent binding of the dyes to the proteins is disclosed by Dubrow, R. S., et al., U.S. Pat. No. 6,475,364, issued Nov. 5, 2002, and by Haugland, R. P., et al., U.S. Pat. No. 5,616,502, issued Apr. 1, 1997. Squaraine dyes, which are dyes based on squaric acid, as well as dyes based on croconic acid, rhodizonic acid, and others, are disclosed by Terpetschnig, E. A., et al., in U.S. Pat. No. 6,538,129, issued Mar. 25, 2003, and in United States Patent Application Publication No. 2005/0202565, published Sep. 15, 2005. The contents of all patents and published literature cited in this specification are hereby incorporated herein by reference.

### SUMMARY OF THE INVENTION

This invention resides in the discovery that certain squaraine dyes are unusually effective as labels for proteins in separation processes where detection is performed by the sensing of fluorescent emissions. These dyes, which associate with the proteins but without covalent binding, produce a fluorescent emission only when the dyes are in the form of association complexes with proteins and polypeptides in the presence of a detergent that forms complexes with the proteins. Detection can thus be achieved by simply incorporating the dyes in a buffered separation medium as an additional solute. Once present, the dyes enable protein/polypeptide detection with high sensitivity and with no detriment to the separation.

### BRIEF DESCRIPTION OF THE FIGURES

FIG. 1a is a trace produced by an Experion™ Automated Electrophoresis System on a sample of a standard set of proteins, using a squaraine dye of the present invention, without baseline correction.

FIG. 1b is a trace produced by the same instrument on the same set of proteins with the same dye as FIG. 1a, but with baseline correction.

FIG. 1c is a plot, taken from the data in FIG. 1a, of the migration time for each protein in the standard vs. the molecular weight of the protein.

FIG. 2a is a trace produced by the Experion™ Automated Electrophoresis System on a sample of rat liver proteins, using a squaraine dye of the present invention.

FIG. 2b is a virtual gel image of the separation shown in FIG. 2a, showing the separated proteins alongside the separation of a standard set of proteins.

FIG. 3 is a plot of peak area at a variety of dilutions of carbonic anhydrase, obtained using a squaraine dye of the present invention.

### DETAILED DESCRIPTION OF THE INVENTION AND PREFERRED EMBODIMENTS

Squaraine dyes that are useful in the practice of this invention include those have the following generic formula 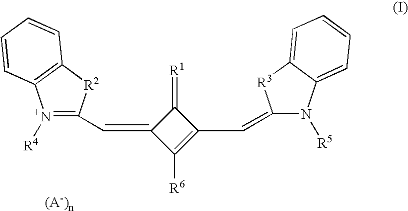
In this formula, and in all other formulas herein where the same symbols appear, the symbols have the following meanings:

R¹ is either O, S, Se, Te, NH, N(C₁-C₄ alkyl), N(aryl), 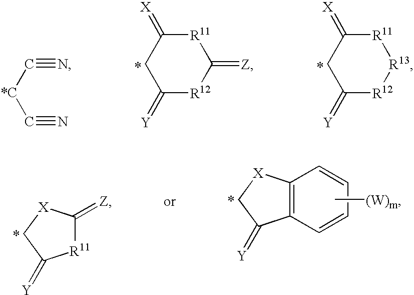
where * denotes the site of attachment in Formula (I); R¹¹ is CH₂, CH(C₁-C₄ alkyl), C(C₁-C₄ alkyl)₂, NH, or N(C₁-C₄ alkyl); R¹² is CH₂, CH(C₁-C₄ alkyl), C(C₁-C₄ alkyl)₂, NH, or N(C₁-C₄ alkyl); R¹³ is CH₂, CH(C₁-C₄ alkyl), or C(C₁-C₄ alkyl)₂; X is O or S; Y is O or S; Z is O or S; W is H or C₁-C₄ alkyl; and m is zero, 1, 2, 3, or 4; and with the proviso stated below in the description of R⁶. The asterisk (*) likewise denotes the site of attachment of any lettered symbol wherever the asterisk is used in this specification and the appended claims.

R² is either O, S, NH, N(alkyl), N(cycloalkyl), N(aryl), C(R²¹)(R²²), or C(R²¹)═C(R²²). When R² is C(R²¹)═C(R²²), the ring moiety shown as a five-membered N-containing ring in Formula (I) to the left of the central cyclobutene moiety becomes a six-membered ring with conjugated double bonds. In the radicals listed in this paragraph, R²¹ and R²², which can be the same or different, are either H or C₁-C₄ alkyl, or together form a single C₃-C₆ alkylene moiety. When R²¹ and R²² together form an alkylene moiety, R² is either a cyclic group sharing a common carbon atom with the adjacent cyclic group shown as a five-membered ring (when R² is C(R²¹)(R²²)) or forms a fused ring structure with the adjacent cyclic group shown as a five-membered ring (when R² is C(R²¹)═C(R²²)). Alkyl, cycloalkyl, and alkylene groups cited in this paragraph are optionally substituted with one or more substituents selected from carboxyl, hydroxyl, and sulfo (i.e., HO—S(O)₂—) groups.

R³ is either O, S, NH, N(alkyl), N(cycloalkyl), N(aryl), C(R³¹)(R³²), or C(R³¹)═C(R³²). When R³ is C(R³¹)═C(R³²), the ring moiety shown as a five-membered ring N-containing in Formula (I) to the right of the central cyclobutene moiety becomes a six-membered ring. In the radicals listed in this paragraph, R³¹ and R³², which can be the same or different, are either H or C₁-C₄ alkyl, or together form a single C₃-C₆ alkylene moiety. When R³¹ and R³² together form an alkylene moiety, R² is either a cyclic group sharing a common carbon atom with the adjacent cyclic group shown as a five-membered ring (when R³ is C(R³¹)(R³²)) or forms a fused ring structure with the adjacent cyclic group shown as a five-membered ring (when R³ is C(R³¹)═C(R³²)). Alkyl, cycloalkyl, and alkylene groups cited in this paragraph are optionally substituted with one or more substituents selected from carboxyl, hydroxyl, and sulfo (i.e., HO—S(O)₂—) groups.

R⁴ is either H, C₁-C₁₂ alkyl, carboxy-substituted C₁-C₁₂ alkyl, hydroxy-substituted C₁-C₁₂ alkyl, sulfo-substituted C₁-C₁₂ alkyl (i.e., HO—S(O)₂-alkyl), phosphono-substituted C₁-C₁₂ alkyl (i.e., (HO)₂P(O)-alkyl), or aryl.

R⁵ is either H, C₁-C₁₂ alkyl, carboxy-substituted C₁-C₁₂ alkyl, hydroxy-substituted C₁-C₁₂ alkyl, sulfo-substituted C₁-C₁₂ alkyl (i.e., HO—S(O)₂-alkyl), phosphono-substituted C₁-C₁₂ alkyl (i.e., (HO)₂P(O)-alkyl), or aryl.

R⁶ is either O⁻, S⁻, Se⁻, Te⁻, NH₂, N(C₁-C₄ alkyl)₂, or N(C₁-C₄ alkyl)(aryl), with the proviso that at least one of R¹ and R⁶ is other than O, S, Se, Te, and anions thereof.

A⁻ is an anion whose presence does not inhibit the adherence of the squaraine dye with proteins and does not prevent the squaraine dye from producing a fluorescence emission.

The index “n” is zero when R⁶ bears a negative charge, and 1 when R⁶ is neutral.

Groups and radicals that are of interest as subgenera of the above symbols in the practice of this invention are as follows. A subgenus of interest for R¹ is O and S, and an individual radical of interest is O. In members of the R¹ class where m is present, m is preferably zero, 1, or 2, and most preferably 1. For R², one subgenus of interest is the optionally substituted methylene group C(R²¹)(R²²). Likewise for R³, a subgenus of interest is the optionally substituted methylene group C(R³¹)(R³²). Further subgenera of interest for R² are those in which R²¹ and R²² are both C₁-C₄ alkyl and are either the same or different, and still further subgenera of interest are those in which R²¹ and R²² are both CH₃. Likewise, further subgenera of interest for R³ are those in which R³¹ and R³² are both C₁-C₄ alkyl and are either the same or different, and still further subgenera of interest are those in which R³¹ and R³² are both CH₃.

A subgenus of interest for R⁴ is C₁-C₁₂ alkyl, carboxy-substituted C₁-C₁₂ alkyl, and hydroxy-substituted C₁-C₁₂ alkyl, a narrower subgenus of interest is C₁-C₆ alkyl, carboxy-substituted C₁-C₆ alkyl, and hydroxy-substituted C₁-C₆ alkyl, a still narrower subgenus of interest is C₁-C₃ alkyl, and an individual radical of interest is CH₃. The same subgenera and radical are of interest for R⁵. A subgenus of interest for R⁶ is O⁻, S⁻, NH₂, N(C₁-C₄ alkyl)₂, and N(C₁-C₄ alkyl)(aryl), a further subgenus of interest is O⁻, S⁻, and N(C₁-C₄ alkyl)₂, a still further subgenus of interest is O⁻ and N(C₁-C₄ alkyl)₂, and individual radicals of interest are O⁻ and N(C₂H₅)₂. An individual anion of interest for A⁻ is

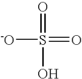

Variations on the structure of Formula (I), included with in the scope of this invention, are those containing one or more halogen substitutions in either or both of the two rings that are shown in the formula as six-membered rings. Preferred halogens are chlorine and bromine. Still further variations are structures in which one or more additional rings are present, fused with either or both of the six-membered rings shown in the formula.

The term “alkyl” is used herein to include both linear and branched alkyl groups, and cyclic and non-cyclic groups. Where the number of carbon atoms in an alkyl group is not stated, the group is not intended to be limited to a particular number, although a range of 1 to 12 carbon atoms is preferred, and a range of 1 to 6 carbon atoms is more preferred. Cyclic alkyl groups are intended to include cyclic groups of 4 to 8 carbon atoms, preferably 5 or 6. The term “aryl” is used herein to denote phenyl and naphthyl groups, with phenyl preferred. The term “independently selected” when preceding a listing of groups or radicals represented by two or more symbols denotes that each symbol can be represented by one group or radical in the list, and that the various symbols can represent either the same radical or group or different radicals or groups, i.e., the selection of a radical or group for one symbol is independent of the selection for another symbol. The term “a” or “an” is intended to mean “one or more.” The term “comprising” when preceding the recitation of a step or an element is intended to mean that the addition of further steps or elements is optional and not excluded.

Examples of individual squaraine dyes within the scope of this invention are as follows:

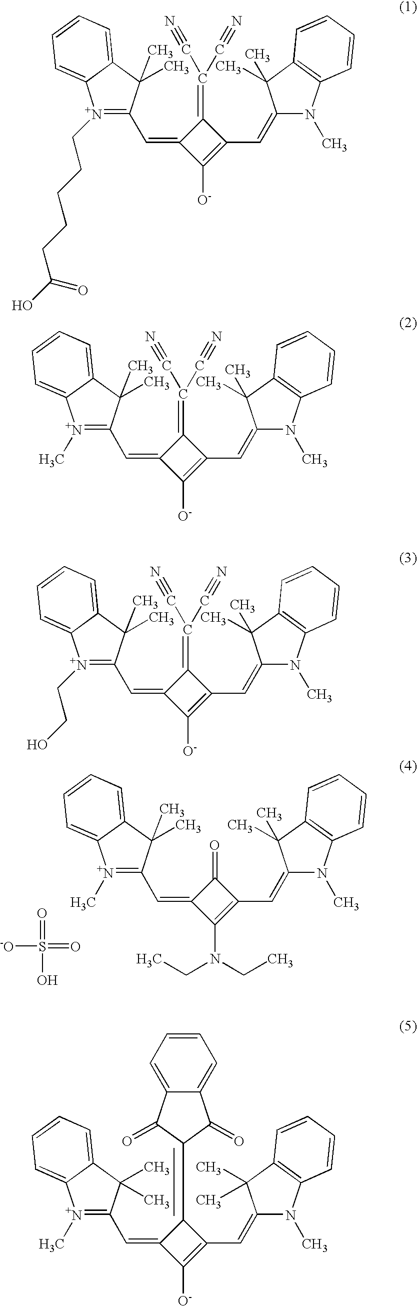

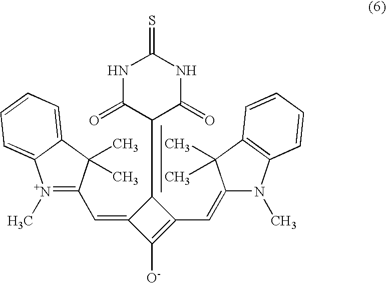

In the practice of this invention, the squaraine dyes of Formula (I) can be used for analyzing a sample to detect proteins or polypeptides therein, in any protein or polypeptide separation procedure where the separation occurs as a result of differences in the migratory behaviors among the proteins or polypeptides. Separation procedures that are of particular interest are capillary electrophoresis and electrophoresis in microfluidic systems. In either of these systems, the separation channel will include the dye, a separation medium, a detergent, a buffering agent, all in a common solution, and optionally a polymeric matrix. The separation medium can be a liquid or a gel. The dye can be placed in the separation medium prior to loading the sample into the system, or it can be added to the sample. The detergent is one that will form complexes with the proteins or polypeptides in the sample. Examples of such detergents are sodium dodecyl sulfate, sodium dodecyl sulfonate, lithium dodecyl sulfate, sodium bis-2-ethylhexyl sulfosuccinate, sodium cholate, perfluorodecyl bromide, cetyltrimethylammonium bromide, didodecylammonium bromide, Triton X-100, polyoxyethylene 10-oleyl ether, polyoxyethylene 10-dodecyl ether, N,N-dimethyldodecylamine-N-oxide, Brij 35, Tween-20, sorbitan monooleate, lecithin, diacylphosphatidylcholine, sucrose monolaurate, and sucrose dilaurate. Anionic detergents are preferred, and examples of anionic detergents are alkyl sulfates and alkyl sulfonates, prime examples of which are sodium dodecyl sulfate, sodium octadecyl sulfate, and sodium decyl sulfate. A particularly preferred detergent is sodium dodecyl sulfate (SDS).

When SDS is used as the detergent, the detergent concentration is preferably from about 0.01% to about 0.5%. Likewise, any buffering agent that is known for use in protein separations can be used. Examples of buffers that are commonly used in SDS-PAGE applications are tris, tris-glycine, HEPES (N-2-hydroxyethyl-piperazine-N-2-ethanesulfonic acid), CAPS (3-(cyclohexylamino)-1-propanesulfonic acid), MES (2-(N-morpholino)-ethanesulfonic acid), Tricine (4-(2-hydroxyethyl)-1-piperazine propanesulfonic acid (EPPS)N-[tris(hydroxymethyl)-methyl]glycine), and combinations thereof. Buffering agents with low ionic strengths are preferred. Examples are zwitterionic buffers, notably amino acids such as histidine and Tricine. Buffering agents that are relatively large ions with relatively low mobilities within the system are also preferred. The concentration of the buffering agent may vary as well, although best results will be obtained in most cases at concentrations of from about 10 mM to about 200 mM, preferably at concentrations from about 10 mM to about 100 mM. When Tris(tris(hydroxy methyl)amino-methane)-Tricine is used as the buffering agent, an appropriate concentration range is from about 20 mM to about 100 mM. An example of a detergent-buffer combination is SDS at a concentration of from about 0.03% to about 0.1% and Tris-Tricine at a concentration of from about 20 mM to about 100 mM, further adjusted if necessary to place the solution at or below the critical micelle concentration (CMC) when operating under the normal operating conditions of the separation process and the separation medium.

In the practice of this invention, the concentration of the detergent is at or below the CMC during the detection of the proteins by fluorescent emission from the dye molecules. This condition of being equal to or below the CMC is achieved by controlling the concentration of the detergent itself, by selecting buffers or buffer additives that affect the CMC, or by controlling both the detergent concentration and the buffer composition. The condition can be maintained during both the separation procedure and the detection, or it can be introduced after separation and before detection by diluting the medium immediately prior to detection. Maintaining the detergent concentration at a level equal to or below the CMC will help minimize background signal from the dye.

The sample to be separated and characterized is typically pretreated with a buffer solution containing the detergent to form complexes of the detergent with the polypeptides (which term will be used herein to include proteins) in the sample. Separately, the squaraine dye is incorporated into the separation medium, which is then placed in a capillary or a microfluidics channel. The detergent-treated sample is then introduced into the capillary or microfluidics channel, typically at one end of the capillary or of a channel segment. An electric field is applied across the length of the capillary or channel, causing the polypeptide-detergent complexes to migrate through the separation medium at rates that vary with the sizes of the complexes, the charges on the complexes, or both. The complexes contact the squaraine dye molecules in the separation medium and upon excitation from an external light source at an appropriate wavelength, only the dye molecules that are inside or in close proximity to a polypeptide-detergent complex produce fluorescence emission, or a level of fluorescence emission that is sufficiently greater than that of dye molecules that are suspended in the bulk of the separation medium. A detection system can thus differentiate between dye molecules associated with a polypeptide-detergent complex and non-associated dye molecules. Thus, there is no need to remove unbound dye molecules from the separation medium before detecting the polypeptides.

When the sample is pretreated with the detergent-containing buffer, the concentration of the detergent in the pretreatment step is preferably greater, on a weight/volume basis, than the polypeptide concentration of the sample, preferably by a factor of at least about 1.4. The detergent concentration in the pretreatment step can range from about 0.5 times to about 3.0 times the detergent concentration in the running buffer, but is preferably less than or approximately equal to, and most preferably less than, the detergent concentration in the running buffer. When SDS is used as the detergent, the detergent concentration in the pretreatment buffer is preferably between about 0.05% and 2%, more preferably between about 0.05% and about 1%, and most preferably less than about 0.5%. If the sample is diluted prior to loading into the separation medium, the detergent level in the loaded sample is preferably between about 0.0025% to about 1%, most preferably from about 0.0025% to 0.5%. All percents herein are weight/volume unless otherwise specified.

A polymeric matrix when present in the separation medium will decrease the mobility of larger polypeptides through the medium relative to smaller polypeptides and thereby increase the degree of separation. A polymeric matrix may also reduce or eliminate electroosmotic flow of the medium within the channel. Any of a variety of polymers, including both cross-linked and gellable polymers, can be used as the polymeric matrix. Non-crosslinked (i.e., “linear”) polymers are preferred in view of the ease by which they can be introduced into capillary and microfluidics channels. Examples of non-crosslinked polymer solutions that are suitable are those set forth in U.S. Pat. Nos. 5,264,101, 5,552,028, 5,567,292, and 5,948,227. The most commonly utilized non-crosslinked polymers are polyacrylamide polymers, preferably a polydimethylacrylamide polymer solution which can be neutral, positively charged or negatively charged. One example is a negatively charged polydimethylacrylamide-co-acrylic acid (as disclosed, for example, in U.S. Pat. No. 5,948,227). The concentration of non-crosslinked polymer can range from about 0.01% to about 30%, preferably from about 0.01% to about 20%, and most preferably from about 0.01% to about 10%. The average molecular weight of the polymer can vary. Samples that require a high polypeptide resolution will require a polymer of relatively high molecular weight, while polymers of lower molecular weight will suffice for less complex separations. In most cases, best results will be achieved with a polymer whose average molecular weight is in the range of from about 1 kD to about 6,000 kD, preferably between about 1 kD and about 1,000 kD, and most preferably between about 100 kD and about 1,000 kD. The polymer can also be selected on the basis of its viscosity. Preferred polymers are those whose viscosity in the separation medium is from about 2 to about 1,000 centipoise, preferably from about 2 to about 200 centipoise, and most preferably from about 5 to about 100 centipoise.

As noted above, the choice of buffering additives is preferably made, and the detergent concentration preferably maintained or adjusted, such that the detergent is at or below, and preferably below, its critical micelle concentration (CMC) at least during the protein or polypeptide detection stage. The CMC is the concentration at which the detergent begins to form independent micelles within the buffer solution to a sufficient degree to interfere significantly with the protein detection. The CMC can also be defined as the highest monomeric detergent concentration, and thus the highest detergent potential, obtainable. As set forth by Helenius et al., in Methods in Enzymol. 56(63):734-749 (1979), the CMC of a detergent solution decreases with increases in the size of the apolar moiety of the detergent molecule and with decreases in the sizes and polarity of polar groups on the molecule. Thus, whether a detergent solution is above or below its CMC is determined not only by the concentration of the detergent, but also by the concentration of other components of the solution that affect the CMC, namely the buffering agent and the total ionic strength of the solution as well as other additives. A variety of methods are known for determining whether a solution is below its CMC. For example, Rui et al., Anal. Biochem. 152:250-255 (1986) disclose the use of a fluorescent N-phenyl-1-naphthylamine dye to determine the CMC of detergent solutions. The concentration of detergent that will place the solution below its CMC can thus be determined experimentally by methods known in the art. Using the squaraine dyes described herein, one can measure the relative micelle concentration in a detergent solution by measuring the fluorescence of the solution as a function of detergent concentration. The CMC is typically indicated by a sharp increase in the fluorescent intensity. Depending on the composition of the separation medium, the CMC will occur in most cases at a detergent concentration between about 0.01% and about 0.5% and a buffering agent concentration between about 10 mM and about 500 mM. Any of the detergents and buffers listed above can be used in the separation medium.

The concentration of the squaraine dye in the separation medium in accordance with this invention can also vary, and is not critical to the novelty or utility of the invention. In most cases, effective results will be obtained at concentrations within the range of about 0.1 μM to about 1 mM, preferably from about 1 μM to about 20 μM.

When the separation is performed by capillary electrophoresis, the capillary can be formed of fused silica, glass or a polymeric material. The buffered separation medium is placed into the capillary channel by pressure pumping or capillary action, and the sample to be separated and characterized is loaded by injection into one end of the capillary channel. As the sample solutes, i.e., the proteins and polypeptides, migrate along the channel under the influence of an electric field, the squaraine dyes associate with the solutes and are detected at a site within the channel toward the cathode end of the channel by a conventional capillary electrophoresis detection system. Additional buffer solutions can be introduced into the flow path of the solutes following their separation, as and if needed, through additional flow paths or capillaries joined to the separation capillary.

When the separation is performed in a microfluidic device, the separation medium is contained in one or more channels of a network of microscale capillary channels disposed within a single integrated solid substrate, typically a laminated structure. In the typical microfluidic device, a separation channel is intersected by at least one sample injection channel. A detector is focused on a locus in the separation channel to detect the separated proteins passing through that locus. Microfluidic devices will most often contain a plurality of sample wells in fluid communication with a common sample injection channel that is in fluid communication with the separation channel to allow multiple samples to be analyzed without cleaning and re-loading the device between samples. Examples of microfluidic devices that can be used in accordance with the present invention are those shown and described in U.S. Pat. No. 6,235,175 issued May 22, 2000. In operation, the separation buffer is first placed in a reservoir and drawn by capillary action into all of the channels of the device, filling the channels. Samples that are to be separated and characterized are separately placed in other reservoirs of the device. Through the energization of appropriate electrodes, the first sample is transported by electrophoretic force from its reservoir through the appropriate channels and intersections to the separation channel. The electrode energization pattern is then changed to cause electrophoretic migration and separation of the sample through the separation channel. While separation of the first sample is occurring, the next sample to be analyzed is transported by selective energization of the appropriate electrodes to a site where it can enter the separation channel, and then separated in the separation channel. The process is repeated for each sample.

Detection of the dye-labeled polypeptides in both capillary systems and microfluidic devices can be performed by optical means in a detection zone within the separation channel. The device is typically transparent, and a detection window can be located at virtually any point along the length of the channel. As the dye-associated solutes pass the detection window, the dye receives a beam of excitation radiation and a fluorescent emission from the dye is detected. An example of a microfluidics device that incorporates components for operation of the electrodes and the detector is the Experion™ Automated Electrophoresis System of Bio-Rad Laboratories, Inc., Hercules, Calif., USA. Another is the 2100 Bioanalyzer from Agilent Technologies, as described in U.S. Pat. No. 5,976,336. Detection methodologies and devices known in the art for fluorescence emissions and for use in capillary electrophoresis and in microfluidic systems can be used.

The following examples are offered for illustration. All experiments reported in these examples were performed using the Experion™ Automated Electrophoresis System referenced above, in conjunction with the Experion™ Pro260 Analysis Kit (also from Bio-Rad Laboratories, Inc.). Those experiments demonstrating the use of a squaraine dye of the present invention did so by substituting the squaraine dye for SYTO™ 60, the dye provided in the kit.

### EXAMPLE 1

The squaraine dye shown above as Formula (6) was dissolved in a solution of 6.75% sodium dodecyl sulfate in dimethyl sulfoxide, to a dye concentration of 140 μM. The resulting solution was used in place of the stain reagent in the Experion Pro260 Analysis Kit, resulting in a separation medium consisting of a buffered polymer solution containing sodium dodecyl sulfate at a final concentration of 0.25% plus the dye at a final concentration 5.2 μM. A set of protein standards selected to form a molecular weight “ladder” consisting of recombinant proteins with molecular weights of 10, 20, 25, 37, 50, 75, 100, 150 and 260 kDa was then used as the sample, and the sample was run through the system according to the standard procedures supplied with the system instructions.

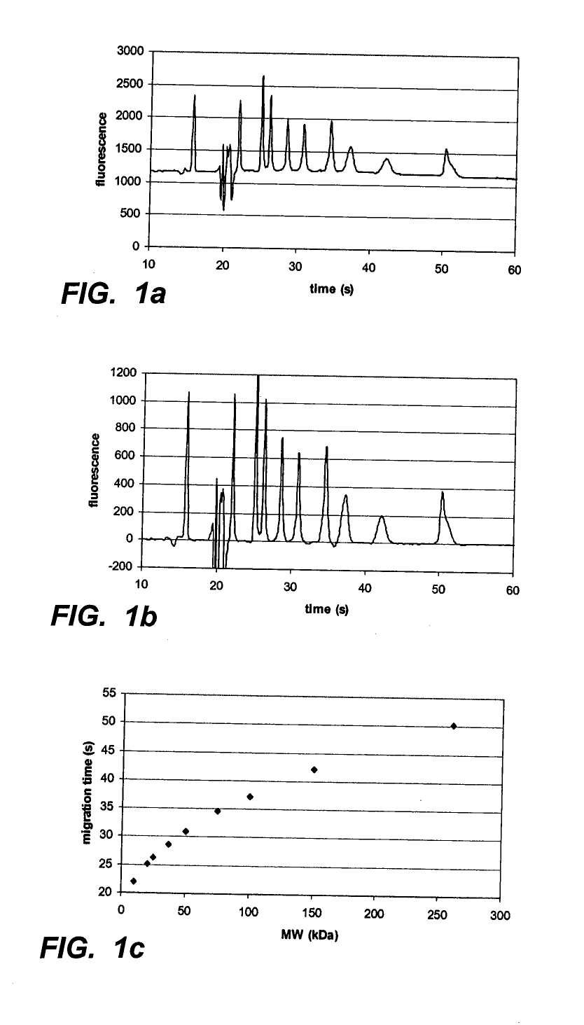

Analyses were performed by use of the software provided by the system, and traces of fluorescence vs. time were generated. The trace of FIG. 1a represents the analysis with the test dye and without the baseline correction provided by the instrument software; the trace of FIG. 1b represents the analysis with the test dye but also with the baseline correction; and the trace of FIG. 1c shows a plot of the migration time vs. the molecular weight for each protein in the set of standards. The data show that the dye functions effectively in rendering the proteins visible, and the migration times of the proteins in the standard set are a smooth function of the protein molecular weight in the presence of the dye.

### EXAMPLE 2

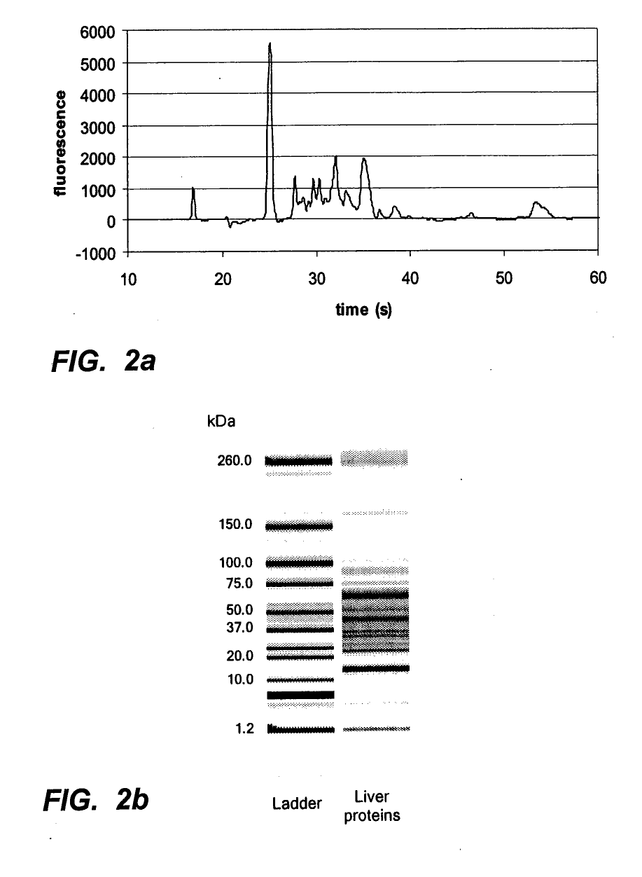

The squaraine dye shown above as Formula (5) was dissolved in a solution of 6.75% sodium dodecyl sulfate in dimethyl sulfoxide, to a dye concentration of 100 μM. The resulting solution was used in place of the stain reagent in the Experion Pro260 Analysis Kit, resulting in a separation medium consisting of a buffered polymer solution containing sodium dodecyl sulfate at a final concentration of 0.25% plus the dye at a final concentration 3.7 μM. A rat liver protein extract was prepared for use as a sample by grinding a sample of frozen rat liver in nine volumes of phosphate-buffered saline plus 1 mM phenylmethanesulfonyl fluoride. The extract was clarified by centrifugation and processed for separation in the Experion System according to the instructions provided with the System. This processed extract was then run through the System according to the standard procedures supplied with the System instructions. The results were analyzed by the System software, and are presented in FIGS. 2a as a trace of fluorescence emission vs. time, and in FIG. 2b in gel-view mode together with the molecular weight “ladder” standard protein mixture supplied by the System. The results indicate that the dye may be used effectively to visualize the microfluidic separation of a complex protein mixture and to estimate molecular weights of the constituent proteins by comparison to the mobility of protein standards.

### EXAMPLE 3

This example illustrates the correlation between fluorescence intensity and protein concentration using a squaraine dye of the present invention.

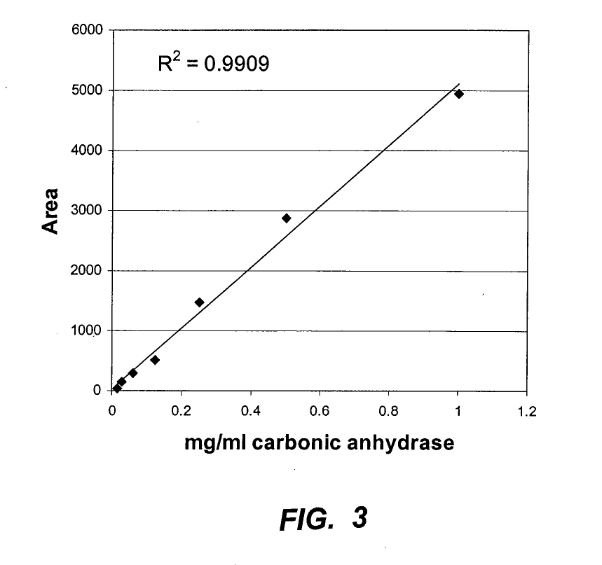

The squaraine dye shown above as Formula (5) was dissolved in a solution of 6.75% sodium dodecyl sulfate in dimethyl sulfoxide, to a dye concentration of 150 μM. The resulting solution was used in place of the stain reagent in the Experion Pro260 Analysis Kit, resulting in a separation medium consisting of a buffered polymer solution containing sodium dodecyl sulfate at a final concentration of 0.25% plus the dye at a final concentration 5.6 μM. In place of the protein mixtures of the preceding examples, a series of dilutions of purified bovine carbonic anhydrase (Sigma-Aldrich) was used as samples. The concentrations of the carbonic anhydrase in the various samples in the series were 1 mg/mL, 0.25 mg/mL, 0.125 mg/mL, 0.06 mg/mL, 0.03 mg/mL, and 0.015 mg/mL. Each dilution was run through the System according to the standard procedures supplied with the System instructions, and the results were analyzed by the System software. A plot of the peak area as determined by the software vs. the concentration of the carbonic anhydrase is shown in FIG. 3, which indicates that the response is proportional to the amount of protein in the sample.

What is claimed is:

1. A method for rendering proteins detectable in an electrophoretic separation process performed in a separation medium that includes a detergent that forms a complex with said proteins, said method comprising including in said medium a photoluminescent compound having the formula: 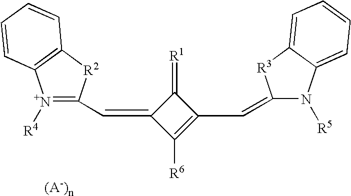
in which:
R¹ is a member selected from the group consisting of O, S, Se, Te, NH, N(C₁-C₄ alkyl), N(aryl), 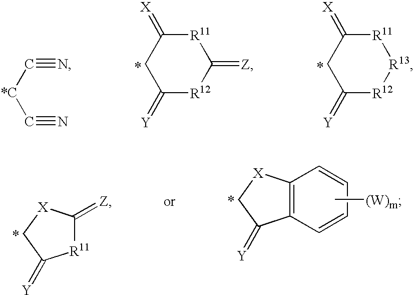
wherein * denotes a site of attachment;
R¹¹ is a member selected from the group consisting of CH₂, CH(C₁-C₄ alkyl), C(C₁-C₄ alkyl)₂, NH, and N(C₁-C₄ alkyl);
R¹² is a member selected from the group consisting of CH₂, CH(C₁-C₄ alkyl), C(C₁-C₄ alkyl)₂, NH, and N(C₁-C₄ alkyl);
R¹³ is a member selected from the group consisting of CH₂, CH(C₁-C₄ alkyl), and C(C₁-C₄ alkyl)₂;
X is a member selected from the group consisting of O and S;
Y is a member selected from the group consisting of O and S;
Z is a member selected from the group consisting of O and S; and
W is a member selected from the group consisting of H and C₁-C₄ alkyl;
m is either zero or an integer of 1, 2, 3, or 4;
R² is a member selected from the group consisting of O, S, NH, N(alkyl), N(cycloalkyl), N(aryl), C(R²¹)(R²²), and C(R²¹)═C(R²²), in which R²¹ and R²² are independently members selected from the group consisting of H and C₁-C₄ alkyl, or together form C₃-C₆ alkylene, said group further consisting of alkyl, cycloalkyl, and alkylene groups substituted with a member selected from the group consisting of carboxyl, hydroxyl, and sulfo;
R³ is a member selected from the group consisting of O, S, NH, N(alkyl), N(cycloalkyl), N(aryl), C(R³¹)(R³²), and C(R³¹)═C(R³²), in which R³¹ and R³² are independently members selected from the group consisting of H and C₁-C₄ alkyl, or together form C₃-C₆ alkylene, said group further consisting of alkyl, cycloalkyl, and alkylene groups substituted with a member selected from the group consisting of carboxyl, hydroxyl, and sulfo;
R⁴ is a member selected from the group consisting of H, C₁-C₁₂ alkyl, carboxy-substituted C₁-C₁₂ alkyl, hydroxy-substituted C₁-C₁₂ alkyl, phosphono-substituted C₁-C₁₂ alkyl, and aryl;
R⁵ is a member selected from the group consisting of H, C₁-C₁₂ alkyl, carboxy-substituted C₁-C₁₂ alkyl, hydroxy-substituted C₁-C₁₂ alkyl, phosphono-substituted C₁-C₁₂ alkyl, and aryl;
R⁶ is a member selected from the group consisting of O⁻, S⁻, Se⁻, Te⁻, NH₂, N(C₁-C₄ alkyl)₂, and N(C₁-C₄ alkyl)(aryl), with the proviso that at least one of R¹ and R⁶ is other than O, S, Se, Te, and anions thereof;
A⁻ is an anion whose presence does not inhibit adherence of said photoluminescent compound with said proteins and does not prevent fluorescence of said photoluminescent compound; and
n is zero when R⁶ bears a negative charge, and 1 when R⁶ is neutral.

2. The method of claim 1 wherein R¹ is a member selected from the group consisting of O and S.

3. The method of claim 1 wherein R¹ is O.

4. The method of claim 1 wherein R² is C(R²¹)(R²²) in which R¹¹ and R¹² are each independently members selected from the group consisting of H and C₁-C₄ alkyl; and R³ is C(R³¹)(R³²) in which R³¹ and R³² are each independently members selected from the group consisting of H and C₁-C₄ alkyl.

5. The method of claim 4 wherein R²¹ and R²² are each independently C₁-C₄ alkyl, and R³¹ and R³² are each independently C₁-C₄ alkyl.

6. The method of claim 4 wherein R²¹, R²², R³¹, and R³² are each CH₃.

7. The method of claim 1 wherein R⁴ and R⁵ are each independently members selected from the group consisting of C₁-C₁₂ alkyl, carboxy-substituted C₁-C₁₂ alkyl, and hydroxy-substituted C₁-C₁₂ alkyl.

8. The method of claim 1 wherein R⁴ and R⁵ are each independently members selected from the group consisting of C₁-C₆ alkyl, carboxy-substituted C₁-C₆ alkyl, and hydroxy-substituted C₁-C₆ alkyl.

9. The method of claim 1 wherein R⁴ and R⁵ are each independently C₁-C₃ alkyl.

10. The method of claim 1 wherein R⁴ and R⁵ are each CH₃.

11. The method of claim 1 wherein R⁶ is a member selected from the group consisting of O⁻, S⁻, NH₂, N(C₁-C₄ alkyl)₂, and N(C₁-C₄ alkyl)(aryl).

12. The method of claim 1 wherein R⁶ is a member selected from the group consisting of O⁻, S⁻, and N(C₁-C₄ alkyl)₂.

13. The method of claim 1 wherein said detergent is sodium dodecyl sulfate.

14. The method of claim 1 wherein said photoluminescent compound is

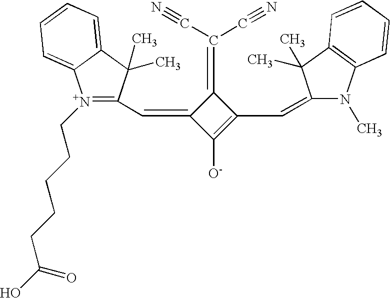

15. The method of claim 1 wherein said photoluminescent compound is

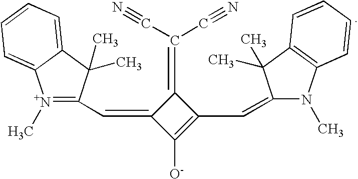

16. The method of claim 1 wherein said photoluminescent compound is

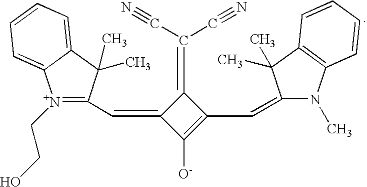

17. The method of claim 1 wherein said photoluminescent compound is

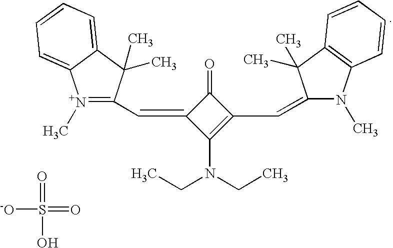

18. The method of claim 1 wherein said photoluminescent compound is

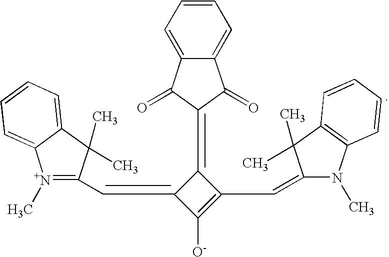

19. The method of claim 1 wherein said photoluminescent compound is [chemical structure]
